(19) United States
(12) Patent Application Publication
(10) Pub. No.: US 2025/0281148 A1
(43) Pub. Date: Sep. 11, 2025

(54) Breast Cancer Screening of Xray-Dense Breasts Using Rotary ABUS and Advanced Artificial Intelligence

(71) Applicant: Shih-Ping Wang, Los Altos, CA (US)

(72) Inventor: Shih-Ping Wang, Los Altos, CA (US)

(21) Appl. No.: 19/218,437

(22) Filed: May 26, 2025

Related U.S. Application Data
(63) Continuation-in-part of application No. 17/243,206, filed on Apr. 28, 2021.
Application No. 17/243,206 is a division of application No. 15/975,544, filed on May 9, 2018, now Pat. No. 11,020,086.
Application No. 15/975,544 is a continuation-in-part of application No. 15/244,950, filed on Aug. 23, 2016, now abandoned.
Application No. 15/244,950 is a continuation of application No. 14/070,336, filed on Nov. 1, 2013, now Pat. No. 9,420,991.
Application No. 14/070,336 is a continuation of application No. 14/076,989, filed on Nov. 11, 2013, now Pat. No. 9,498,184.
Application No. 14/076,989 is a continuation of application No. 14/077,050, filed on Nov. 11, 2013, now abandoned.
Application No. 14/070,336 is a continuation-in-part of application No. 13/296,023, filed on Nov. 14, 2011, now Pat. No. 8,579,819.
Application No. 14/076,989 is a continuation-in-part of application No. 13/296,023, filed on Nov. 14, 2011, now Pat. No. 8,579,819.
Application No. 14/077,050 is a continuation of application No. 13/296,023, filed on Nov. 14, 2011, now Pat. No. 8,579,819.
Application No. 13/296,023 is a continuation of application No. 11/513,481, filed on Aug. 30, 2006, now abandoned.
(60) Provisional application No. 60/713,282, filed on Sep. 1, 2005.
(60) Provisional application No. 61/769,913, filed on Feb. 27, 2013.
(60) Provisional application No. 61/769,913, filed on Feb. 27, 2013.
(60) Provisional application No. 62/503,869, filed on May 9, 2017.
(60) Provisional application No. 62/647,813, filed on Mar. 25, 2018.

(51) Int. Cl. A61B 8/08; A61B 8/00; A61B 8/14

(52) U.S. Cl. CPC A61B 8/0825; A61B 8/14; A61B 8/4209; A61B 8/4218; A61B 8/4455; A61B 8/4461; A61B 8/4494; A61B 8/463; A61B 8/483; A61B 8/54; A61B 8/145; A61B 8/4281; A61B 8/4405

(57) ABSTRACT

An ultrasound system for breast cancer screening scans a breast by rotating a dish-shaped template with one or more breast-facing recesses and a smoother nipple covering area about a center or rotation that is at the breast but is spaced from the nipple, to produce a multiplicity of radially oriented two-dimensional images, and speeds up assessment of the images by subjecting them to an artificial intelligence process that identifies likely abnormalities in the breast as volumes of interest (VOIs) and characterizes each VOI as benign, suggestive of a follow-up sooner than a normal interval, or likely cancerous, and computer-generates a report directing the patient accordingly, wherein the process includes comparing volumes of suspected abnormalities estimated from a current scan with volumes of corresponding regions of the breast from one or more previous scans.

### REFERENCE TO RELATED APPLICATIONS

This application is a continuation in-part (CIP) of U.S. patent application Ser. No. 17/243,206 filed Apr. 28, 2021.

Ser. No. 17/243,206 is a division of Ser. No. 15/975,544 filed May 9, 2018 (now U.S. Pat. No. 11,020,086 issued Jun. 1, 2021).

Ser. No. 15/975,544 is a CIP of Ser. No. 15/244,950 filed Aug. 23, 2016 (abandoned).

Ser. No. 15/244,950 is a continuation of each of (a) Ser. No, 14/070,336 filed Nov. 1, 2013 (now U.S. Pat. No. 9,420,991 issued Aug. 23, 2016), (b) Ser. No. 14/076,989 filed Nov. 11, 2013 (now U.S. Pat. No. 9,498,184 issued Nov. 22, 2016), and (c) Ser. No. 14/077,050 filed Nov. 11, 2013 (abandoned).

Ser. No. 14/070,336 is a CIP of Ser. No. 13/296,023 filed Nov. 14, 2011 (now U.S. Pat. No. 8,579,819 issued Nov. 12, 2013).

Ser. No. 14/076,989 is a CIP of Ser. No. 13/296,023 filed Nov. 14, 2011 (now U.S. Pat. No. 8,579,819 issued Nov. 12, 2013).

Ser. No. 14/077,050 is a continuation of Ser. No. 13/296,023 filed Nov. 14, 2011 (now U.S. Pat. No. 8,579,819 issued Nov. 12, 2013).

Ser. No. 13/296,023 is a continuation of Ser. No. 11/513,481 filed Aug. 30, 2006 (abandoned).

This application claims the benefit of Provisional Application No. 62/647,813 filed Mar. 25, 2018; Provisional Application No. 62/503,869 filed May 9, 2017, Provisional Application No. 61/769,913 filed Feb. 27, 2013; and Provisional Application No. 60/713,282 filed Sep. 1, 2005.

This application incorporates by reference the entire content of each of the foregoing patent applications and claims the benefit of the filing date of each.

### FIELD

This patent specification pertains to systems and methods for automated ultrasound screening of patients' breasts and to automated assessment of ultrasound scan results to identify lesions and those among them that are likely cancerous using rotary scanning and artificial, using rotary ultrasound scanning and artificial intelligence (AI).

The system and method are particularly important for patients with breasts that are dense to x-rays and therefore may not benefit sufficiently from x-ray mammography or other x-ray modalities.

### BACKGROUND AND SUMMARY

The patents, published patent applications, and other publications referenced in this patent specification are hereby incorporated by reference in their entireties. Several publications are by shortened names following by numerals in parenthesis and are fully cited in the list of references at the end of the specification, before the claims.

An object of the system and method described below is to achieve ultrasound breast scanning and utilization of ultrasound measurements that are uniquely efficient and cost-effective. The disclosed embodiments achieve such results by scanning a patent's breast that preferably is oriented upward, e.g. with the patient supine of on her side, with a rotary template that rotates one or more ultrasound transducers around the breast and preferably has (a) ribs or depressions that ensure that the breast is held stable but is not under uncomfortable overall pressure and (b) a relatively smooth central area to cover and protect the breast's nipple, and processing the ultrasound measurements in ways that preferably include comparing the current volume of a suspected region with the volume from a previous scan and categorizing suspected abnormalities to thereby conclude that a suspected region does not currently require further examination, or requires another scan soon, e.g., in a few months, or is sufficiently indicative of cancer to require further examination, e.g. by other imaging modalities such as MRI and/or biopsy or surgery. In this manner, the vast majority of screening scans can be immediately dismissed as not indicative of cancer, while the few remaining patients can be directed to another scan soon or to immediate work-up and/or medical care, thus making screening much more efficient and cost-effective.

According to Global Cancer Statistics 2020 by Sung et al (1), breast cancer is now the leading cancer with over 2.6 million new cases per year causing more than 680,000 deaths per year, which makes it the biggest cancer killer of women. Approx. 40% to 50% of these women have x-ray dense breasts. According to a study by Webb et al (2), most breast cancer deaths are associated with lack of screening. This is a particularly serious matter for younger women due to faster cancer cell doubling rate, where a delay in detection, even by less than a year, would likely put the cancer into later stages with much lower survival rates and higher medical treatment costs. Webb's study further found that 49 is the medium age of breast cancer death, by age of initial diagnosis. If this study could be extrapolated, it would mean more than 20,000 per year in the U.S. and 340,000 per year worldwide young women, who were initially diagnosed with breast cancer under age 50, would die of breast cancer. In the meantime, the standard age to start screening in Europe and the U.S. is age 50. This suggests that screening practice should change, although the change can only be successful by overcoming many hurdles.

A significant portion of these young women could be saved by screening but only if a practical and low-cost screening modality becomes available. Both practical and low-cost are important because the detectable breast cancer in this age group is very low, approx. 2 cancers/1000 screening examinations. A recent CISNET study by Monticciolo et al (03) shows that by starting to screen at age 40 instead of age 50, the breast cancer mortality reduction could be significantly improved (by 25.4% to 30% if screened biannually, 41.7% if annually). Indeed, the authoritative recommendation for starting age of breast cancer screening by USPSTF (United States Preventative Service Task Force) recently changed from age 50 to 40. X-ray mammography is known for being less effective in dense breasts, and thus US-FDA also started a mandate for dense breast notification in September 2024. This would lead to large scale supplemental screening of 20-40 million additional women with dense breasts in the U.S.

Two supplemental screening modalities, Breast MRI and breast ultrasound, have been approved by US-FDA. Breast MRI is still too expensive for mass screening, and the contrast medium used in that procedure can cause adverse events in patients. So far, the use of breast MRI has been limited to higher risk and diagnostic patients. Handheld ultrasound is US-FDA approved for screening dense breasts but, although low in cost, is tedious and requires well-trained operators. ABUS (automated breast ultrasound scanner) has been available since 2012 but has not become a satisfactorily practical and low-cost solution at least in part because assessing the 1,000 of more 2D ultrasound images per breast by a highly trained physician reader and the required patient positioning slow down the patent throughput rate too much. ABUS is commercially offered by Hitachi for scanning a prone patient. See https://social-innovation.hitachi/en-us/solutions/life_economy/sofia-3d/. Another ABUS is offered by Delphinus, for scanning a prone patient's breast immersed in a liquid. See https://www.delphinusmt.com/softvue/how-it-works. Rectilinear scan ABUS scanning supine patients is offered by GE HealthCare is a little faster in patient throughput. See GEHC's Invenia at https://www.gehealthcare.com/products/ultrasound/breast-ultrasound/invenia-abus?srsltid=AfmBOorBkkiunU72IDw4Pc46HG-UkSYycSfE_G09K2tvNC7C5OBLVvJV. A commercially available AI has been available for the rectilinearly scanning Invenia/ABUS. This AI is known as QV-AI and is a powerful deep-learning AI developed by QView Medical (https://www.qviewmedical.com/). Simple AI (artificial intelligence), based on few 2D slice images, was recently proposed. See for example Shen et al (8). Artificial intelligence system reduces false-positive findings in the interpretations of breast ultrasound exams, Nature Communications (13).

To screen the 20-40 million young women in the U.S. and several hundred million young women worldwide with dense breast, the screening systems should be significantly lower in both capital cost and operating costs than the known systems commercialized to date. It should be easy to operate by less skilled operators, easily coupled with advanced AI, and readily accessible to patients, such as in rural areas via mobile vans, easy to reach shopping malls, etc. These requirements are even more important in countries outside of U.S. The Rotary ABUS for supine patients, as further described in the following, aims to meet such needs.

Another, an even more challenging hurdle is that a new screening modality would encounter a lengthy “learning curve” issue. The time needed to train new physician readers for the new screening modality is many years long. The “learning curve” issue for x-ray screening mammography has been well studied, conducted on 231 radiologists for over 20 years. See Miglioretti et al (10) . . . . The article reports that it would take as many as five years of training/practice for a physician to be sufficiently proficient to achieve a 10% recall rate (generally minimum accepted false positive call back rate), in the interpretation of mammography images. Basically, it means that it is very difficult to learn to safely dismiss a cancer look-like suspicious lesion without calling back the patient for additional, expensive workup diagnostic examinations. Because of the “learning curve” issue, it has been observed that the adoptions of all new screening modalities have been slow. Time to reach competency in reading ABUS results can be even more of a challenge as a typical supine ABUS screening like GEHC's Invenia/ABUS would generate approx. 1,000 2D images per breast, while screening x-ray mammography would only generate two 2D images per breast. Since detectable breast cancer in dense-breast women is approx. 2 per 1000 examinations, this means the physician reader for a commercialized system such as Invenia/ABUS would have to review approx. 1,000,000 2D images to catch one cancer and at the same time would have to safely dismiss 900 to 940 benign cases per 1000 examinations (to maintain a reasonable recall rate of 6-10% or less).

To screen 20-40 million dense breast women per year in the U.S., at an average physician reading speed of 2,000 to 4,000 examinations per year, would mean training perhaps 10,000 to 20,000 additional physician readers, which would likely take decades. Therefore, just to have a low-cost ABUS is not enough without employing advanced AI, based on “deep Learning” technology, to significantly reduce the need to train so many physician readers. “Deep Learning” technology will be discussed in more detail further below in this specification.

Another hurdle is in the reimbursement. Physician readers are currently paid at the same rate for reading a case of ABUS exam as a case of mammography exam. However, an ABUS exam containing 500 times more images than a mammography exam would normally take significantly longer in reading time. Therefore, physician readers would prefer to learn reading mammography cases to earn more, over learning to read ABUS cases.

In view of the “learning curve” and reimbursement issues and the anticipated slow availability of trained physician readers, a solution to screening a great number of dense breasts is to employ advanced AI. Advanced AI can perform as an autonomous reader to largely replace a need for human readers at the same or better sensitivity. So far, no advanced AI for autonomous read of ultrasound images has been cleared by US-FDA or regulatory agencies in other countries. Nearly 25 years ago, the inventor of this patent application introduced the first AI commercially for screening x-ray mammography. This earlier AI has been used mainly as the second reader to aid human readers. The broad use of advanced AI based on “Deep Learning” technology in recent years has progressed to the very promising autonomous reading by AI. One such study is by Yala et al (04), which shows nearly 20% of the mammograms could be safely triaged away without further human reading. Another more recent study by Pedemonte et al (14) shows nearly 42% of the mammograms could be safely triaged away without further human reading. More studies in Europe are in progress with the goal of replacing the required human second-reader in the European double-reading screening mammography system.

Several clinical studies have been published related to QV-AI in aiding the reading of Invenia/ABUS (GEHC's supine rectilinear ABUS) images, which were mostly directed to reduce the long ABUS image reading time per examination, reduce obvious oversights, give the physician reader more confidence while helping to accelerate the learning curve process. In a 2018 Whitepaper by QView, where QV-AI was then called QVCAD (see www.qviewmedical.com/press; file:///C:/Users/IvanKavrukov/Downloads/Breast%20cancer%20saving%20more%20lives%20-%20Nov%202018.pdf), the FROC (free receiver operator characteristic) curve shows a sensitivity of 85% at Operating point 1 with (1-Spec) of 0.1 FP/frame. Since Invenia/ABUS generates 6 frames (sets of approx. 300 2D images each frame) per examination, this means Operation Point 1 is at a specificity of 0.53 (=(1-0.1)⁶). This means QV-AI can remove 53% of the benign cases autonomously at a sensitivity of 85%, which is equal or better than the sensitivity of radiologists. In 2024, the latest release of QV-AI, based on larger training cases for its Deep Learning algorithm, is capable of operating at over 90% sensitivity and 67% specificity, allowing autonomous removal of ⅔ of the benign cases. This means the existing physician readers need not have to read ⅔ of the benign cases-leading to an increase in the human reader's patient throughput by 3-fold. This further means the number of physician readers needed to be trained can be reduced by a factor of 3 and thus accelerate the adoption of Invenia/ABUS. For the screening clinic, this would mean a 3-fold increase in patient load and 3-fold increase in revenue, although having to increase the installed ABUS units by 3-fold.

Since QV-AI algorithm acts on volumetric data from Invenia/ABUS, and since Rotary ABUS also constructs acquired 2D scan images into a 3D volume for AI computation, QV-AI can be applied to Rotary ABUS use as well.

A detectable cancer rate of approx. 2 per 1,000 examinations per year means that approx. 99.8% of the screening cases are benign and that autonomous read could realistically reach over 90 to 94% or more removal rate (the typical 6 to 10% physician recall rate), which means that screening with ABUS+AI would eventually not need human readers to achieve a lower recall rate. The screening results for likely non-benign cases can be directly sent as needed to human physician operated facilities for further diagnostic workup examinations.

For further improvements in AI, in addition to training the AI on much larger number of cancer and benign cases through deep-learning processes combined with manual assists, one key direction is to make use of the seldom considered fact that breast cancer cell doubling rate of younger women is very fast. Very few studies were devoted to breast cancer cell doubling rates, especially for young women under age 50. Approx. 150-200 days is the generally recognized doubling rate for all ages. A good study that included young women under the age of 50 is by Peer et al (05). The Peer et al study provides the medium breast cancer cell doubling time (with 95% range) for 3 age groups: (a) under age 50, (b) age 50-70, and (c) over age 70, respectively 80 days (44-147 days), 157 days (121-204 days), and 188 days (120-295 days). Or, respectively, 0.22 year (0.12-0.4 years), 0.43 year (0.33-0.56 years), and 0.52 year (0.33-0.81 years), ranging from 1.2× volume doublings to 8× volume doublings per year. In view of such large measurable changes in volume with time, vast improvements in both sensitivity and specificity of the autonomous reading AI can be made by comparing temporal volume changes, from two different or consecutive screenings, of suspicious lesions. As further discussed later and shown in FIG. 5 and FIG. 6, temporal comparison of lesion diameters is much less sensitive and less reliable than temporal comparison of lesion volume. This is in part because suspicious lesions are frequently irregular in shape and their average diameter measurement critically depends on the plane from which the diameter is selected. Furthermore, the average diameter changes only 26% when the volume doubles.

The cancer cell doubling rates are so fast that, as discussed later, the medium Sojourn time of under 50 younger women is barely one year. Therefore, the screening interval should preferably be 6 months, which should be set at much shorter time interval than the medium Sojourn time, to thereby make it more effective in saving lives. Screening every 6 months would mean that the total number of screening examinations per year would be doubled while the cancer detection rate would now drop to one cancer per 1,000 examinations. This increased screening cost could be best accomplished effectively with a practical low-cost Rotary ABUS, preferably supine, in combination with advanced AI, which has lower capital and operating costs than approaches such as the rectilinear scanning in Invenia/ABUS. This is because Rotary ABUS has higher patient throughput rate (by almost 3-fold), can use less trained operators, shorter cleaning time after each examination, and can use much cheaper shorter ultrasound transducers.

Rotary ABUS as improved with AI read can significantly reduce or even eliminate the use of x-ray related modalities for younger women and the attendant radiation risks. The current x-ray dose for x-ray related modalities is not modest—each examination is about equivalent to standing at less than 2 miles from Hiroshima epicenter. Feig and Hendrick (6) pointed out that radiation risk on younger women is an order of magnitude higher than that for older women. Feig and Hendrick further estimated that x-ray radiation used by mammography would cause one death for every 37.5 lives saved by mammography. Therefore, for screening below age 50, where low-cost screening modality is a necessity (since detectable cancer rate is much lower than for older women plus a need to screen in shorter intervals) and with higher sensitivity to radiation, ultrasound is the best screening modality.

Since ABUS can collect volumetric data, it is practical to implement volume change comparisons between 2 screenings. Several tumor volume change measurement algorithms are known. For example, an automatic registration-based algorithm for direct measurement of volume change in tumors has been published by Sarkar et al (7). Since Rotary ABUS performs only one scan per breast, while Invenia ABUS performs 3 or more scans per breast, Rotary ABUS would be more accurate in location and registration.

This patent specification describes systems directed to overcoming many of the challenges described above by using rotary type ABUS (automated breast ultrasound scanner) together with an advanced AI (artificial intelligence) to screen dense-breast patients, preferably in the supine position, to achieve a practical screening that is low-cost, both in capital costs and operating costs, and suited for broad adoption. High percentages (50% to 99+%) of basically benign cases can be triaged away using advanced AI, as an autonomous reader, before human physician involvement, to answer the global need for a practical and low-cost screening system and method for women in age range of 30-60, many if not most of which have dense breasts, without waiting years to train thousands of physician readers needed to interpret the screening images. This can address the challenges of rapid cancer cell doubling rate, especially in young women to significantly improve sensitivity and specificity through applying temporal volume comparison technique on successive screenings.

Embodiments of supine Rotary ABUS described in this patent specification include improvements over those reported in U.S. Pat. No. 11,020,086 and in published U.S. application 2021/0244383 A1. One important new feature in the new system described in this patent specification is that preferably there is no nipple hole in the rotating template, and another is that the position of the scan center preferably is moved to a new location up from the nipple (toward the patient's head). As the new rotating template can be free of noticeable ridges, there is no “nipple bump” during scanning. This makes placement of the scanner relative to the breast less critical, which makes it easier for less experienced operators. Furthermore, as shown in FIG. 3, the center of the Rotary ABUS is shifted above the nipple to cover more of the upper quadrants of the breast, where most of the breast cancers are found.

In the following specification, a low-cost and easy to operate ultrasound screening system and method for dense breasts are presented. This system and method combine an advanced AI system with a Rotary ABUS, with the patient preferably in the supine position.

The term advanced AI (AAI) as used in this patent specification is defined below in connection with FIGS. 10, 11 and 12.

According to some embodiments an ultrasound system for breast cancer screening comprises: a rotary scanner comprising a rotary template, a radially oriented ultrasound transducer, a motor, and an acoustically transmissive membrane, wherein the rotary scanner is configured to scan a patient's breast by contacting a scan area of the breast with the template and transducer through the membrane and rotating the template and transducer with the motor relative to the membrane and the breast; the rotary scanner has one or more breast-facing ribs and recesses between ribs and is configured to carry out the scan while exerting pressure on the breast through the membrane that is plural times lower per unit area at portions of the scan area remote from the transducer than under the transducer and further has an area that is relatively smooth and is configured to overlie the breast's nipple during the scan; and the scan produces ultrasound results for a multiplicity of radially oriented two-dimensional (2D) planes in the breast; a computer-implemented processor configured to process the ultrasound results by applying thereto algorithms configured to find one or more abnormalities and define them as three-dimensional (3D) volumes of interest (VOI) and to further divide the identified VOIs into (i) cancer-suspicious VOIs and (ii) at least one category of VOIs that are not cancer-suspicious, wherein the algorithms include comparing volumes of interest from a current scan with volumes of interest from one or more previous scans of the breast; and a processing facility configured to receive at least ultrasound results for cancer-suspicious VOIs and to subject the ultrasound results for the cancer-suspicious VOIs to further computer processing and examination to identify cancerous VOIs.

According to some embodiments, the system further includes one or more of: (a) the one or more breast-facing ribs and recesses are circumferentially spaced from the transducer and each other, whereby breast portions facing the one or more recesses experience the lower pressure per unit area; (b) the one or more recesses comprise plural recesses that are circumferentially spaced from the transducer and from each other by breast-facing ribs with rounded surfaces; (c) the area of the template that is relatively smooth is free of the one or more ribs and recesses; (d) the template is configured to rotate about a center of rotation spaced toward the patient's head from the breast's nipple; (e) the center of rotation is at the upper outer quadrant of the breast being scanned; (f) the breast-facing surface of the template converges to the center of rotation of the template at an angle of no more than 15 degrees; (g) the membrane and template are uninterrupted by a nipple hole; (h) the rotary scanner is configured to scan an upwardly facing breast; (i) the transducer protrudes toward the breast from a majority of the breast-facing surface of the template, whereby breast portions that are in the scan area but are remote from the transducer experience said lower pressure per unit area; (j) the computer-implemented processor is further configured to divide the VOIs that are not cancer-suspicious into (a) likely benign VOIs, (b) VOIs for follow-up; (k) the computer-implemented processor is configured to carry out the dividing through an artificial intelligence process; (I) the comparing of volumes include comparing the volumes of respective pairs of VOIs that match in 3D location in the breast; (m) the rotary template is essentially flat at a surface therein facing the breast except for one or more shallow recesses that are spaced from the ultrasound transducer; (n) a ring-shaped support for the mesh and ring-shaped, foam-like material over a breast-facing surface of the ring-shaped support; (o) the rotary scanner includes a window and both the window and the rotary template are sufficiently transparent to enable visualization of the breast from outside the rotary scanner; (p) the rotary template is transparent and the rotary scanner includes an imaging module configured to view the breast through the rotary template and the system further includes a display configured to display images of the template and breast taken with the imaging module; and (q) the rotary template is shaped as a shallow dish converging to the center of rotation at an angle less than 15 degrees.

According to some embodiments, an ultrasound system for breast cancer screening comprises: a rotary scanner comprising a rotary template, one or more radially oriented ultrasound transducers, a motor, and an acoustically transmissive membrane, wherein the rotary scanner is configured to scan a patient's breast by contacting the breast with the template and the one or more transducers through the membrane and rotating the template and the one or more transducers with the motor relative to the membrane and the breast such that the one or more transducers maintain acoustic contact with the breast over a dish-shaped path; the template has a breast-facing surface with one or more recesses from the dish-shaped path and an area that is free of the recesses and is configured to overly the breast's nipple while the template rotates over the breast; and the scan produces ultrasound results for a multiplicity of two-dimensional (2D) planes in the breast; a computer-implemented processor configured to process the ultrasound results by applying thereto algorithms configured to identify likely abnormalities as three-dimensional (3D) volumes of interest (VOI) and to divide the identified VOIs into (i) cancer-suspicious VOIs and (ii) at least one category of VOIs that are not cancer-suspicious; and a processing facility configured to receive ultrasound results for cancer-suspicious VOIs and to subject the ultrasound results for the cancer-suspicious VOIs to further computer processing and observation to identify cancerous VOIs; wherein at least one other computer-implemented processor and the processing facility compares volume of a VOI from a current scan with that of a similarly located VOI from a previous scan of the breast.

According to some embodiments, the system described in the immediately preceding paragraph further comprises one or more of: (a) the rotary template has one or more breast-facing rounded ribs that are circumferentially spaced from the transducer and from each other and define said one or more recesses, whereby breast portions facing the one or more recesses experience lower pressure per unit area than breast portions that are under the one or more transducers and ribs; (b) a breast-facing area of the template that is free of the one or more recesses also is free of a nipple hole; (c) the center of rotation of the template is at the upper outer quadrant of the breast being scanned; (d) the breast-facing surface of the template converges to the center of rotation of the template at an angle of no more than 15 degrees; (e) the algorithms that the computer-implemented processor is configured to apply are artificial intelligence algorithms; (f) a ring-shaped support for the mesh and ring-shaped, foam-like material over a breast-facing surface of the ring-shaped support are included; and (f) the patient's breast faces up during the scanning.

According to some embodiments, a method of ultrasound screening for breast cancer comprises: scanning a patient's breast with a rotary template and one of more ultrasound transducers that together rotate over the breast while the one or more transducers maintain acoustic coupling with the breast through a membrane to produce two-dimensional (2D) ultrasound images for respective notional planes through the breast; subjecting the 2D ultrasound images to an artificial intelligence (AI) process configured to identify likely abnormalities in the breast as volumes on interest (VOIs) and to assess the VOAs to produce AI results that characterize each as (i) benign VOI, (ii) VOI for follow-up, or (iii) likely cancerous VOI, wherein the AI process includes comparing a volume of one or more of the identified VOIs with the volume of a corresponding one or more VOI from one or more previous scan of the breast if available; computer-generating the AI results to automatically produce a patient report that (i) directs a patient with a VOI assessed as likely cancerous to work-up medical procedures to further characterize the likely cancerous VOI, (ii) directs a patient with a VOI assessed as follow-up to an ultrasound screening after a time interval related to the patient's age and medical history and different for different age groups, and (iii) directs a patient with a VOI assessed as benign to an ultrasound screening after a time interval that also is related to the patient's age and medical history but is longer than for a patient with a benign VOI.

According to some embodiments, the method further comprises one or more of: (a) including a physician review of ultrasound results relating to a VOI that the AI process has characterized as likely cancerous to confirm the VOI as likely cancerous or change its characterization to benign or follow-up, and wherein the step of computer-generating a patient report directs the patient based on the results produced in the physician review; (b) the computer-generating of a patient report is configured to direct patients to follow-up ultrasound screening based on a table relating patient age to statistical rate of doubling of VOI volumes; and (c) the computer-generating of a patient report is configured to direct patients with a VOI for follow-up and are under 50 years of age to a follow-up ultrasound screening in less than 6 months.

According to some embodiments, a rotary scanner comprises a rotary template, a radially oriented ultrasound transducer, a motor, and an acoustically transmissive membrane, wherein; the rotary scanner is configured to scan a patient's breast by contacting a scan area of the breast with the template and transducer through the membrane and rotating the template and transducer with the motor relative to the membrane and the breast; the rotary scanner template has one or more breast-facing recesses and is configured to carry out the scan while exerting pressure on the breast through the membrane that is plural times lower per unit area at portions of the scan area remote from the transducer than under the transducer and further has a smoother central area for nipple placement.

According to some embodiments, the rotary scanner described in the immediately preceding paragraph further comprises: (a) a rotary template that is spherically curved, with a radius of curvature equal to 1-3 times the diameter of the rotary template, or (b) a rotary template is conically curved, with a less than 15° angle.

### BRIEF DESCRIPTION OF THE DRAWING

To further clarify the above and other advantages and features of the subject matter of this patent specification, specific examples of embodiments thereof are illustrated in the appended drawings. It should be appreciated that these drawings depict only illustrative embodiments and are therefore not to be considered as limiting the scope of this patent specification or the appended claims, and that features shown on one drawing can be used in place of or in addition to features depicted in another. The subject matter hereof will be described and explained with additional specificity and detail through the accompanying drawings in which:

FIG. 1 is a perspective view of a rotary ABUS scanner, according to some embodiments.

FIG. 2 is an exploded view of components of a rotary ABUS scanner, according to some embodiments.

FIG. 3 is a schematic view of a rotary ABUS scanner over a patient's left breast, according to some embodiments.

FIG. 4A shows a top view of a rotary template, FIG. 4B shows a cross-sectional view of the template when spaced from a breast, and FIG. 4C shows a cross-sectional view when the template is in contact with a patient's breast, according to some embodiments.

FIGS. 5A-C are bottom and cross-section views of a rotary template containing shallow recessed areas between rounded ribs, according to some embodiments.

FIGS. 5D-F are bottom and cross-section views of a rotary template containing a single shallow recessed area, according to some embodiments.

FIG. 6 is a Table of breast cancer cell doubling rate for 3 age groups, based on published data by Peer et al (5).

FIG. 7 shows a volumetric image cylinder constructed from 2D scanned images as well as an example of 2D scanned image with AI-detected suspicious lesions, according to some embodiments.

FIG. 8 is a screen shot of a patient breast scan obtained with a prototype ABUS operating at 8.9 MHz and 39 fps.

FIG. 9 is a screen shot of a 2D image for display, formed by combining two 2D image obtained at 180° apart.

FIG. 10 is a process flow chart for screening asymptomatic patients using a combined Rotary ABUS and AI screening system in a high patient throughput medical center, according to some embodiments.

FIG. 11 is a process flow chart for screening asymptomatic patients using a combined Rotary ABUS and AI screening system in a simple, AI-centric, no-physician medical center, according to some embodiments.

FIG. 12 is a process flow chart for screening asymptomatic patients using a combined Rotary ABUS and AI screening system in a medical center, according to some further embodiments.

FIG. 13 is a block diagram illustrating aspects of an AI screening system suitable for use with a Rotary ABUS system, according to some embodiments.

### DETAILED DESCRIPTION

A detailed description of examples of preferred embodiments is provided below. While several embodiments are described, the new subject matter described in this patent specification is not limited to any one embodiment or combination of embodiments described herein, but instead encompasses numerous alternatives, modifications, and equivalents. In addition, while numerous specific details are set forth in the following description to provide a thorough understanding, some embodiments can be practiced without some or all these details. Moreover, for the purpose of clarity, certain technical material that is known in the related art has not been described in detail to avoid unnecessarily obscuring the new subject matter described herein.

Individual features, components, and elements of one or several of the specific embodiments described herein can be used in or in combination with other described embodiments or with other features, components, and elements. Like reference numbers and designations in the various drawings indicate like elements.

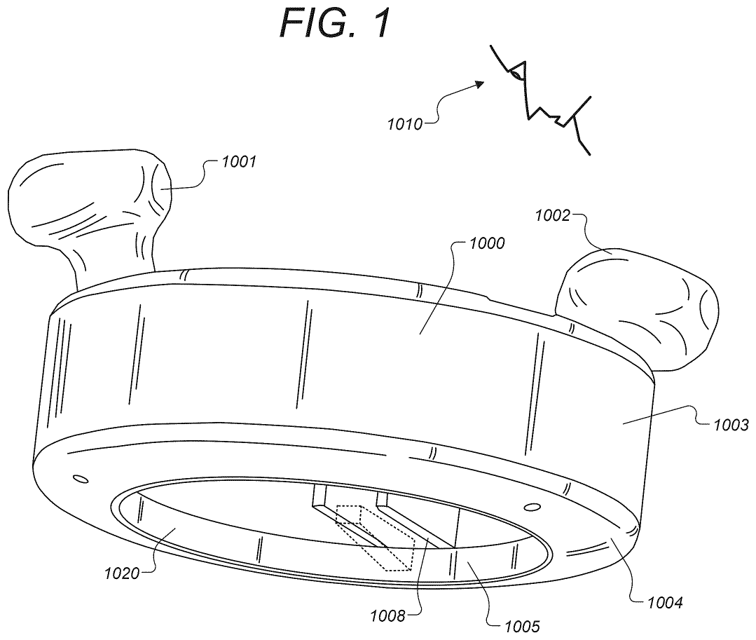

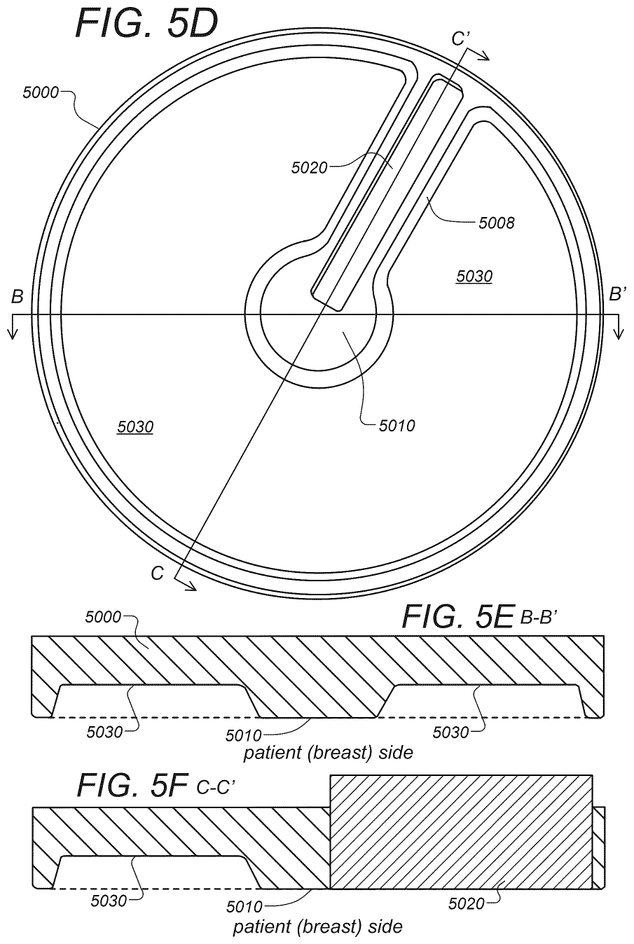

FIG. 1 shows an example of an assembled Rotary ABUS Scanner 1000. An operator 1010 such as a medical technician or a physician can look at the patient's breast through a transparent rotating template 1020 for positioning and quality check of ultrasonic gel wetting or contact between rotary template 1020 and the patient's breast. FIG. does not show an optional top, transparent dome cover, but such a dome cover can be added to keep the whole instrument better looking. A camera and a display screen can be added to aid the operator by providing good instant images for better positioning and breast contact inspection, as described in U.S. Pat. No. 11,020,086. Two easy-to-hold handles 1001 and 1002 are at the top of the Rotary ABUS Scanner 1000 for the operator to hold during positioning and the scanning procedure, in which the patient preferably is facing up, in the supine position, on a table. Other forms of handles and support arm(s), anchored to a wall or to an ultrasound machine support gantry can be used, for example as illustrated in FIG. 6 of U.S. Pat. No. 11,020,086. Plural Rotary scanners 1000 in different sizes for different size breasts can be added to supporting arms. The active diameter of the Rotary ABUS preferably varies from 10 cm to 20 cm. This is the diameter of the area scanned by the ultrasound transducer as described further below. A diameter that should fit most patients is roughly 15 cm. FIG. 1 shows a holder 1008 for one ultrasound transducer. However, plural transducers can be used concurrently to reduce the cost of using one large transducer for larger diameter scanners. The Rotary ABUS scanner 1000 has an outer shell 1003 and a front circumferential cover 1004. The front cover 1004 holds a stationary fine mesh screen 1005 configured to contact chestwardly and help hold the patient's breast during the scanning process as well as to facilitate good ultrasonic contact between the ultrasound transducer and the patient's breast. Ultrasonic gel is typically applied to the breast and the mesh screen. The mesh screen 1004 is typically used just once per examination and is discarded after the examination. The mesh screen 1004 can be replaced by a thin and preferably transparent plastic sheet, such as mylar film, or another barrier film or sheet, but mesh is preferred for better ultrasonic contact and fewer scan artifacts. As will be further described, the rotating template 1020 preferably is a solid, preferably transparent plastic piece with no nipple hole. A significant advantage of a Rotary ABUS with a solid rotating template, compared to systems such as the Invenia/ABUS, is that it keeps ultrasonic gel from getting inside the ABUS scanner 1000 and makes cleaning much easier after each examination. In FIG. 1, the holder 1008 holds an ultrasound transducer shown in dotted lines in holder 1008.

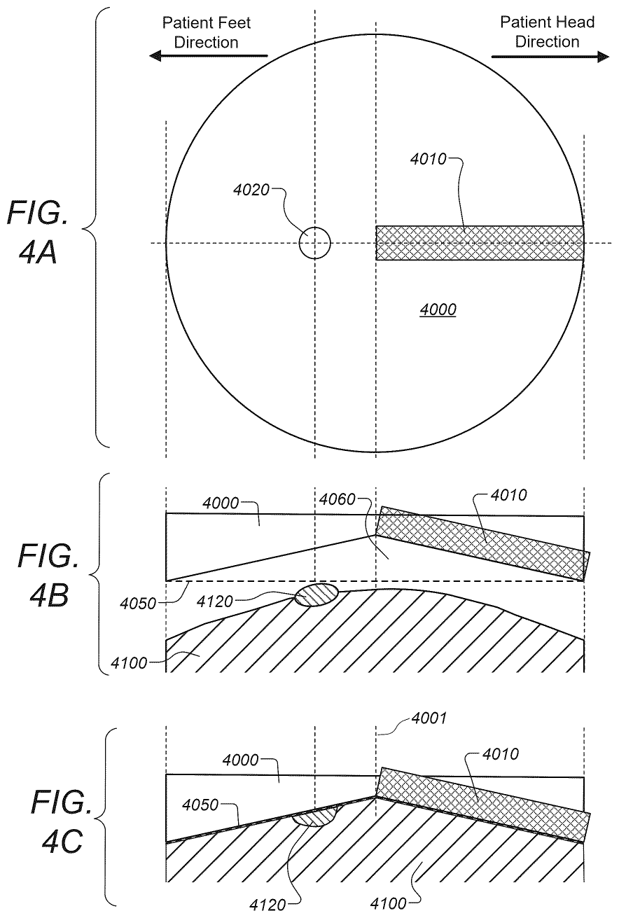

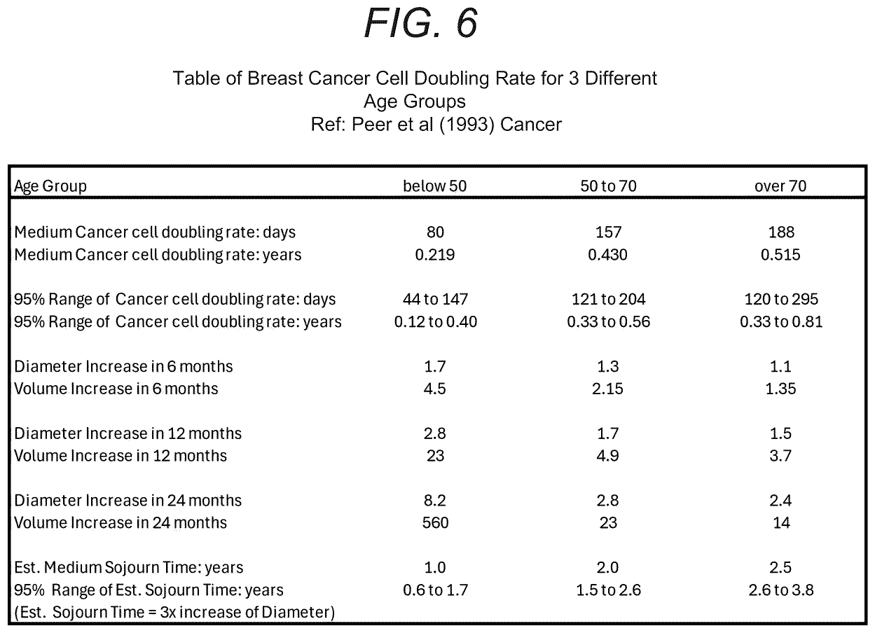

Rotary scanner 1000 can be a part of a larger system, for example as illustrated in FIG. 6 of parent U.S. Pat. No. 11,020,086, incorporated herein in its entirety by reference, which shows a scanner 101 as a part of a system that includes supports for the scanner, a patient table, cart 620 that houses an ultrasound engine, and a display 630. Scanner 1000 is configured to contact a patient's breast chestwardly with rotary template 1020 carrying a radially oriented ultrasound transducer (4010 in FIG. 4) and to scan the breast while rotating the template and transducer relative to the breast about a center of rotation offset, preferably toward the patient's head, from the breast's nipple (4001 in FIG. 4) to thereby produce ultrasound results for a multiplicity of notional two-dimensional (2D) planes (e.g., 710, 7020, and 7030 in FIG. 7). A computer-implemented image processor (4003 in FIG. 4 that can be like processor 622 in U.S. Pat. No. 11,020,086, incorporated herein by reference) is configured to process the results for the 2D planes to identify likely abnormalities as three-dimensional (3D) volumes of interest (VOI), such as VOIs 7012, 7022, and 7032 in FIGS. 7 and 1022 in U.S. Pat. No. 11,020,086, and to divide the VOIs into (i) cancer-suspicious VOIs and (ii) at least one category of VOIs that are not cancer-suspicious. The term cancer as used here means malignant. The system can further include a processing facility (4005 in FIG. 4) configured to receive at least ultrasound results for cancer-suspicious VOIs and to subject the ultrasound results for selected 2D planes and/or 3D results derived therefrom to further computer processing and observation to identify cancerous VOIs.

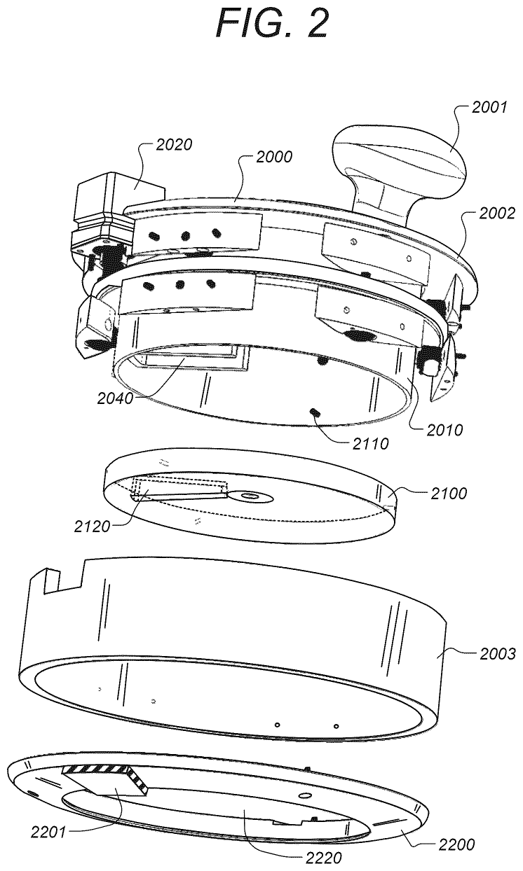

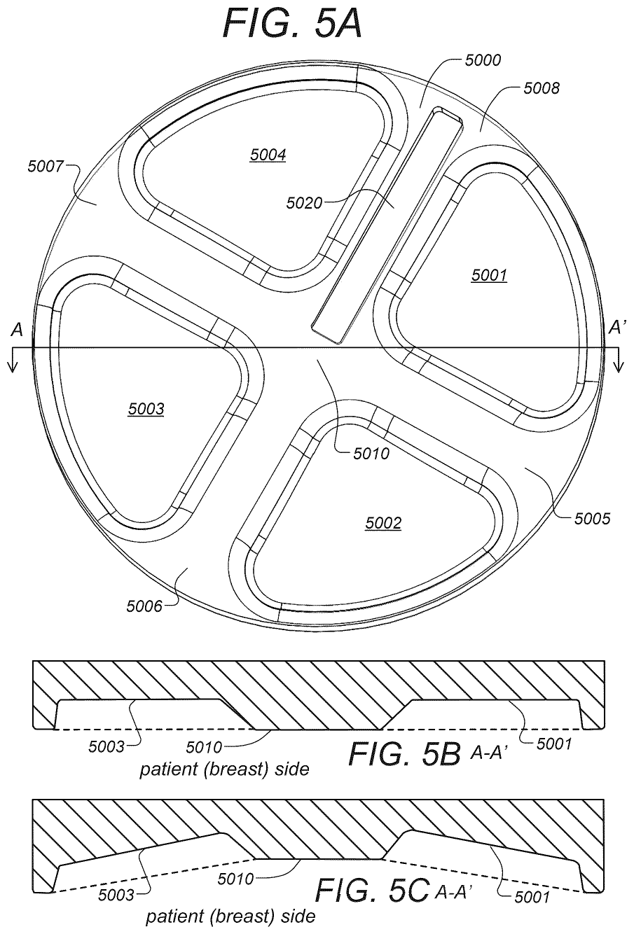

FIG. 2 shows components of a Rotary ABUS scanner 2000 in an exploded view. Holder 2001 is attached to a top cover piece 2002. An outer cover 2003, shown in this figure as detached from the top cover 2002, is lowered to expose rotating parts. The rotary motion is provided by a rotating cylinder 2010, which has circular gear teeth on its top rim (hidden in this view). The rotating cylinder 2010 is driven by a motor 2020. The rotating cylinder 2010 has a transducer holder 2040 to hold an ultrasound transducer (4010 in FIG. 4). This transducer holder 2040 can alternatively be installed on the upper side of a rotating template 2100, instead of on the rotating cylinder 2010. The rotating template 2100, shown here as a detached piece, is normally attached to the rotating cylinder 2010, using a few horizontally located screws such as 2110. The rotating template 2100 has slot 2120 for the ultrasound transducer (not shown) so that the transducer can be held with its scanning surface roughly flush with the bottom surface of the rotating template and in contact with patient's breast through a non-rotating mesh screen 2220 or another membrane material. A front, non-rotating cover plate 2200 is shown at the bottom of FIG. 2, detached from the outer cover 2003. This front plate 2200 carries a mesh screen 2220 that stays stationary relative to the breast during the rotary scanning motion to help hold the patient's breast during the scanning motion. The mesh screen 2220 is normally used just once per examination and is discarded after the examination. Adding a softer material 2201, such a foam ring, to the outside of the periphery of the front cover 2200 facing the breast or providing a softer foam-like cover over the rim of front cover 2200, can avoid or at least significantly reduce patient discomfort that may result from contact with a hard-material rim of front cover 2200. FIG. 2 shows only a small arc of soft material 2201 but preferably the soft material is in the form of a ring around the entire periphery of front plate 2200. FIGS. 5A-5F, which are discussed below, show additional or alternative ways to improve patient comfort, using a rotary template having recessed areas.

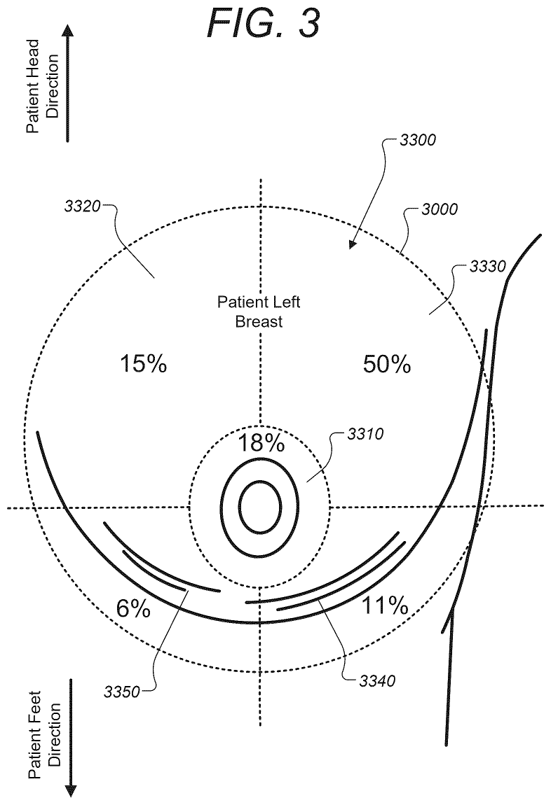

FIG. 3 schematically illustrates Rotary ABUS scanner over a patient's left breast 3300. The scan circle 3000 represents the circumference of the area scanned by the ultrasound transducer of the Rotary ABUS scanner. This patent specification refers to the area in circle 3000 as the scanned area. The breast is notionally divided into 4 quadrants, with the nipple 3310 at the center of these quadrants: 3320 is the upper inner quadrant, 3330 is the upper outer quadrant, 3340 is the lower outer quadrant, and 3350 is the lower inner quadrant. The percentage figure in each quadrant represents a generally accepted probability of breast cancer occurrence rate at that area of the breast-respectively, 18% for the nipple area 3310, 15% for the upper inner quadrant 3320, 50% for the upper outer quadrant 3330, 11% for the lower outer quadrant, and 6% area 3320, 15% in the upper inner quadrant 3030, 11% in the lower outer quadrant 3340, and 6% for the lower inner quadrant 3350. The typical positioning of the Rotary ABUS scanner is designed for good coverage of the breast portions where cancer is most likely to be found, by preferably placing the center (4001 in FIG. 4C) of the rotary scan motion higher than the nipple, toward the patient's head. The nipple need not be in the center of the scan. Experiments with a prototype Rotary ABUS scanner constructed as described in this patent specification have confirmed that, with the stationary mesh holding the breast during the scan, nipple bumps during the scans can be avoided and so a nipple hole in the scanning template can be eliminated. This arrangement makes the positioning much less critical, and the device can be operated by much less trained, lower cost operators.

The location of the breast quadrant in which cancer is found, for example the upper outer quadrant, should be entered into the patient record. This information is useful and used during deep-learning training of advanced AI.

The Rotary ABUS scanner template 2100 can be provided in several sizes in scan circle diameter ranges such as 10 cm, 15 cm, 20 cm, and even larger or in between sizes, much like bra sizes, to provide a good fit for each size breast. The Rotary ABUS scanner need not cover all or even most of the axilla area of the breast. A handheld transducer or a small 3D volume scanner, for example as offered by Hologic, Inc. of Marlborough, MA for elastography ultrasound imaging, can be added to the system to cover axilla and other areas as needed or desired. Advanced AI can also be applied to the scanned images acquired by this auxiliary scanner.

FIGS. 4A-4C illustrate further design and positioning considerations, with the patient's head (not shown) to the right. FIG. 4A is a top view showing a rotating template 4000 with a transducer 4010. Also shown is the position of the nipple 4020. Notably, the center of rotation 4001 of the template 4000 preferably is above the nipple and closer to the patient's head, to increase coverage of the breast's upper quadrants for better cancer detection. The patient's head and feet directions are shown in FIG. 4A and these directions also apply to FIGS. 4B and 4C.

FIG. 4B is a sectional side view of rotating template 4000, transducer 4010, and of the position of a stationary mesh 4050 before contacting the patient's breast. The patient's breast 4100 with the breast nipple 4120 are shown below and not yet in contact with stationary mesh 4050. Transducer 4010 is mounted in or through a transducer slot in rotating template 4000 such that the transducer's active surface can be roughly flush with the bottom surface of the rotating template and can make direct contact with the patient's breast through the stationary mesh 4050 during scanning, with good acoustic coupling. The contact with the patient's breast typically is assisted with ultrasonic gel. The rotating template can be in conical shape, with an optimum angle 4060 of preferably less than 15°, i.e., the template is nearly flat. This patent specification refers to a template with such angle or a smaller angle as essentially flat. A benefit of such a small angle is that a higher ultrasound frequency can be applied to obtain a better, higher spatial resolution image with an almost flattened breast compressed by an almost flat rotating template, to achieve the thinnest breast that ultrasound needs to penetrate and image. The rotating template 4000 and the transducer 4010 can both have a spherically curved surface, with equal curvature, for better fit with the breast and enable greater patient comfort. The radius of curvature of the breast-facing surface of the template preferably is in the range of 1 to 10 times or more the diameter of the rotating template. For best patient comfort, a radius of curvature of 1 to 3 times the diameter of the rotating template is optimal.

The rotary template 4000 can be slightly curved such that its side facing the breast conforms to a shallow spherical shape or otherwise curved shape, with a radius or curvature one to three times the diameter (or the largest dimension) of the template, to thereby provide greater patient comfort and better contact between the ultrasound transducer and the breast. The transducer also can be curved to approximately match the template curvature.

FIG. 4C is a sectional side view but shows the rotating template 4000 of the Rotary ABUS scanner in contact with the patient's breast 4100 through mesh 4050, with the breast nipple 4120 located off the center of the scan center of rotation. The stationary mesh 4050 now contacts the breast 4100 and helps hold it in place during the scan. The scanned area of the breast in this case is dish-shaped.

Typical handheld ultrasound transducers for breast scan typically require no more than about 0.5 kg of pressure on the breast. This is sufficient to achieve good ultrasonic contact and reduces artifacts. However, in rotary ABUS, rABUS, the pressure felt by the breast preferably is not the same everywhere in the scanned area of, e.g. a 15 cm diameter area. In rABUS, the pressure than areas of the template outside the transducer exert on the breast is much less than the pressure that the ultrasound transducer exerts while scanning, e.g. approximately 50 or more times less. Thus, rABUS can have a rotary template that delivers the needed pressure for its transducer and at the same time applies much less pressure through the membrane on most of the remainder of the breast. For example, the transducer pressure on the breast outside the transducer can be less than 1 kg, such as 0.5 kg or even less. If the breast-facing area of the transducer is 10 cm square and the pressure that the transducer exerts on the breast is 1 kg, this means that the pressure per square cm on the breast under the transducer is 0.1 kg. If the pressure that the template exerts on the breast through the membrane outside the transducer area is the same 0.1 kg per cm square, and that area is 100 times larger, this means that the pressure outside the transducer area would be 10 kg. To avoid patient discomfort due to high pressure on the breast, the rABUS scanner is configured such that the pressure at most of the scan area outside the current position of the scanning transducer is much less per unit area than under the current position of the transducer. The pressure outside the current position of the transducer can be just enough for membrane (mesh) 4050 to hold the breast in place during rotary scanning; for example, the overall pressure outside the transducer area can be 1 kg or less. Rotary templates having hollow recessed designs can be used to reduce the perceived applied pressure. Several such designs are shown in FIGS. 5A to 5F. A less preferred alternative to achieve this difference in perceived pressure on the breast during scanning is to have the transducer protrude slightly toward the breast from the breast-facing surface of the membrane contacting the breast, such as by a mm or so or a few mm.

When performing a handheld ultrasound study on the breast, the applied pressure typically ranges from 1 to 2 pounds (approximately 0.5 to 1 kilogram). This level of pressure ensures good contact between the transducer and the skin, displaces air to avoid artifacts, and enables adequate imaging without undue discomfort or tissue distortion. The exact pressure may vary depending on factors like the patient's anatomy, the area being examined, and the sonographer's technique. In sensitive areas like the breast, sonographers are trained to use the lowest effective pressure that enables obtaining high-quality images and thereby enhance patient comfort. The contact area of a handheld ultrasound transducer used for breast imaging typically ranges from about 4 to 16 cm², depending on the size and type of the probe. For example, Linear Array Transducers (commonly used for breast imaging) are 3-5 cm long and 1-3 cm wide and thus contact the breast over a Contact Area of approximately 6-12 cm². The primary reason for applying pressure with a handheld ultrasound transducer is to ensure good acoustic coupling between the transducer and the skin with the help of ultrasound gel, which is critical for accurate imaging. This is important for the following reasons: (1) Elimination of Air Gaps: Air is a poor conductor of ultrasound waves because it has a low acoustic impedance compared to soft tissue. Even tiny air pockets can cause significant reflection and scattering of the ultrasound waves, leading to artifacts. Adequate pressure eliminates these air gaps, ensuring the ultrasound gel fully fills the space between the transducer and the skin. (2). Reducing Artifacts: Insufficient contact pressure can cause several types of artifacts, including: (i) Reverberation artifacts: Multiple reflections between the transducer and an air pocket; (ii) Dropout or shadowing artifacts: Areas that appear dark due to poor transmission of sound waves; and (iii) Edge artifacts: Where sound waves bend at the interface of poorly coupled areas. Suitable transduce pressure on the breast during scanning should provide Consistent Tissue Interface by gentle but firm pressure that helps flatten the skin and superficial tissue layers, creating a uniform interface for the sound waves to travel through. This reduces variability and improves the clarity of structures being imaged.

FIG. 5A is a bottom view of a rotating template 5000 containing shallow recessed areas facing the patient's breast. It has been discovered that in rABUS with a diameter larger than 10 cm, better results with less frequent artifacts and greater patient comfort can be achieved by using a rotating template 5000 that is a solid, preferably transparent plate with one or preferably several shallow recessed areas 5001, 5002, 5003, and 5004 separated by ribs 5005, 5006, 5007 and 5008 that transition to the recesses with smooth rounded rib walls. The recess depth preferably is in the range of 2 mm to 40 mm and more preferably in the range of 5 mm to 20 mm, and the total area of the recession preferably is in the range of 40% to 80% of the area scanned with the ultrasound transducer through the membrane (mesh) 4050. The circumferential distance between adjacent recesses can be, for example, from a few mm to a few cm. The slot for the transducer is shown as 5020. To avoid nipple bumps during the scan, which can cause artifacts and distortion in the scan images as well as patient discomfort, the template preferably has a sufficiently large smooth central area 5010 to ensure that the patient's nipple can be easily positioned inside this smooth center area 5010. The area of the smooth portion 5010 preferably is in the range of 2 cm to 8 cm in diameter, depending on the diameter of rABUS, but the diameter of the central smooth area 5010 can be more if desired. An added advantage of the shallow recesses is reduced friction between the rotary template 5000 and the membrane (mesh) 4050 during scanning the breast with the ultrasound transducer. A template with such recesses also is perceived as more comfortable by patients, with less overall pressure selected by the operator. The recesses allow portions of the breast that extend up into the recesses during scanning to be less compressed than the portion of the breast under the transducer during the scan. This beneficial feature can also be employed in prone type rotary ABUS, such as those commercialized by Hitachi under the trade name Sofia, where patient discomfort has been reported due to high perceived breast compression.

FIG. 5B shows a cross-sectional view of an embodiment where the rotary template is a flat plate with shallow recesses and a central smooth area for the placement of the nipple.

FIG. 5C shows the cross-section view of an embodiment where the rotary template is slightly conical shaped, with shallow recesses and a central smooth area for the placement of the nipple.

FIG. 5D is a bottom view of a rotating template 5000 containing a single shallow recessed area 5030 facing the patient's breast and taking up most of the scanned area. In these embodiments, the transducer 5020 is supported by the template. The template 5000 includes a single rib 5008 surrounding the transducer. In this example the number of ribs is reduced. FIGS. 5E and 5F are cross section views along B-B′ and C-C′ in FIG. 5D, respectively.

The Rotary ABUS is designed to acquire enough high quality 2D images to support advanced AI and provide a combined low-cost screening system and method. Preferably the 2D image slice thickness, or distance between notional adjacent image slice planes at or near the radial periphery of the scanned area, is in the range of 0.1 to 2.0 mm. The most preferable range is 0.25-0.5 mm image slice thickness near the circumference of the scanned area. This translates, for a rotary ABUS with a 15 cm active diameter (the diameter of the scanned area), into a range of 1,000 to 2,000 2D images per breast, with much finer slice spacing in the central regions of the scan area. For a typical scan time of one minute per breast for 2000 2D images, the frame rate can be comfortably set at 35 fps (frames per second).

FIG. 6 shows a table on breast cancer doubling rates for 3 age groups, computed from the data published by Peer et al (5). According to Peer et al, the medium breast cancer cell doubling time (with 95% range) for 3 age groups is: under age 50, age 50-70, and over age 70, respectively 80 days (44-147 days), 157 days (121-204 days), and 188 days (120-295 days). Or respectively 0.22 year (0.12-0.4), 0.43 year (0.33-0.56), and 0.52 year (0.33-0.81), ranging from 8 doublings to 1.2 doublings per year. From this data, medium volume (diameter) increases in 6 months for the 3 age groups are respectively 4.5× (70% increase for diameter) for the under 50 age group, 2.15× (30% for diameter) for the 50-70 age group, and 1.35× (10% increase) for the over 70 age group. Likewise, for 12 months interval, the respective medium volume (and diameter) growth are respectively 23× (2.8× diameter) for the under 50 age group, 4.9× (1.7×) for the 50-70 age group, and 3.7× (1.5× for diameter) for the over 70 age group. Notably, the increase in volume is much more than the increase in diameter. The table is reproduced below:

| Age Group | below 50 | 50 to 70 | over 70 |
|---|---|---|---|
| Medium Cancer cell doubling rate: days | 80 | 157 | 188 |
| Medium Cancer cell doubling rate: years | 0.219 | 0.430 | 0.515 |
| 95% Range of Cancer cell doubling rate: days | 44 to 147 | 121 to 204 | 120 to 295 |
| 95% Range of Cancer cell doubling rate: years | 0.12 to 0.40 | 0.33 to 0.56 | 0.33 to 0.81 |
| Diameter Increase in 6 months | 1.7 | 1.3 | 1.1 |
| Volume Increase in 6 months | 4.5 | 2.15 | 1.35 |
| Diameter Increase in 12 months | 2.8 | 1.7 | 1.5 |
| Volume Increase in 12 months | 23 | 4.9 | 3.7 |
| Diameter Increase in 24 months | 8.2 | 2.8 | 2.4 |
| Volume Increase in 24 months | 560 | 23 | 14 |
| Est. Medium Sojourn Time: years | 1.0 | 2.0 | 2.5 |
| 95% Range of Est. Sojourn Time: years | 0.6 to 1.7 | 1.5 to 2.6 | 2.6 to 3.8 |
| (Est. Sojourn Time = 3x increase of Diameter) | | | |

Sojourn time can be defined as the time interval that a barely detectable cancer takes to grow to palpable size, or diagnostic size. Typically, this is the time interval for a cancer lesion to grow say from 5-7 mm in diameter to 15-20 mm, which is estimated to be the growth time interval for somewhere around a 3× increase in diameter (or 27× increase in volume). As shown in FIG. 6, the estimated medium Sojourn times (and estimated 95% range) for the 3 age groups are respectively, 1 year (0.6 to 1.7 years) for the under 50 group, 2.0 years (1.5 to 2.6 years) for the 50-70 group, and 2.5 years (2.6 to 3.8 years) for the over 70 group. To be desirably effective, the screening interval should be significantly shorter than the Sojourn time. This means that for the under 50 age group, screening interval should be set at around 6 months or even shorter. For the under 50 age group, biannual screening (24 months interval) setting, where the medium volume growth would be 560 times (medium diameter 8.2 times), is clearly ineffective. It is very surprising that USPSTF would overlook the fast cancer doubling factor and recommended biannual screening for age 40 to 50 age group. As shown in FIG. 6, a 6-month screening interval can benefit all 3 age groups, as the volume increases are respectively 4.5×, 2.15× and 1.35×, which can be reliably detected with currently available volume comparison algorithms. This points to a way to vastly improve the performance, both in sensitivity and specificity, of AI by adding temporal volume comparison of suspicious lesions from consecutive screenings. By shortening the screening interval to 6 months, the number of screening examinations per year would double in the U.S. and quadruple in Europe (where biannual screening is the current standard). This would place even more emphasis on the need for a practical low-cost ABUS system with an advanced AI system preferably operating in an autonomous mode with high sensitivity and specificity.

FIG. 7 illustrates a cylindrical volume 7000 constructed from 2D images of a breast scanned with a rotary scanner, where the z-direction is perpendicular or at least transverse to the patient's chest. Also shown as examples are three 2D scan images, respectively 7010, 7020 and 7030. Three suspicious lesions are detected by AI, one each in the three 2D scanned images, respectively 7012, 7022 and 7032, with respective diameters 15 mm, 10 mm and 5 mm as shown. The two larger suspicious lesions are in the typical detection range and can be seen by or shown to a physician reader. The smallest lesion 7032, with 5 mm diameter, is below the typical detection size and typically is not seen by or shown to a physician reader but may be shown if so required or desired.

By adding temporal volume comparison algorithm to AI, any noticeable increase in volume of any suspicious lesion, between consecutive examinations, increases its suspiciousness. In one scenario, if all 3 suspicious lesions detected by AI in a current examination scanned images are also found in the prior examination images and all have approx. same volumes, then the improved AI (AAI) can determine that all 3 suspicious lesions are benign and need not flag them to the attention of a physician reader. This would improve the detection specificity of the AI and reduce the potential expensive recall rate. This feature is very important when AAI is used in autonomous mode, since the increased specificity increases the percentage of benign cases to be removed or dismissed before reading by human physician readers is required.

In another scenario, if only the 2 larger suspicious lesions detected by AI in the current examination scanned images are also found in the prior examination images and had approx. same volumes, then the improved AI would determine that the 2 larger suspicious lesions are benign and need not flag them to the attention of the physician reader. If the smallest lesion was not detected in the prior examination scan or was smaller in volume in the prior examination, then this suspicious lesion will be flagged to the attention of the physician reader. This would improve the cancer detection sensitivity of AAI. This feature is also very important when AAI is used in autonomous mode, since the AAI preferably should operate at much higher sensitivity than physician readers. Excess sensitivity can also be traded for increased specificity by operating at a point further to the left on the ROC curve for Rotary ABUS and FROC curve for rectilinear ABUS (such as the Invenia system).

If a shorter annual screening interval, such as every 6 months, is applied to younger women under age 50, then adding temporal volume comparison to the AAI would be very significant. According to FIG. 6, in 6 months, the medium cancer volume would grow by a very measurable factor of 4.5.

As discussed above in the BACKGROUND AND SUMMARY section, an early form of advanced AI, developed by QView Medical, Inc, when used in the form of autonomous first read (AFR), can remove up to 67% of the normal screening cases at 90% sensitivity. Adding temporal volume comparison significantly increases both specificity and sensitivity. This is especially significant for lifesaving in dense breast young women whose cancer cell doubling time is so short.

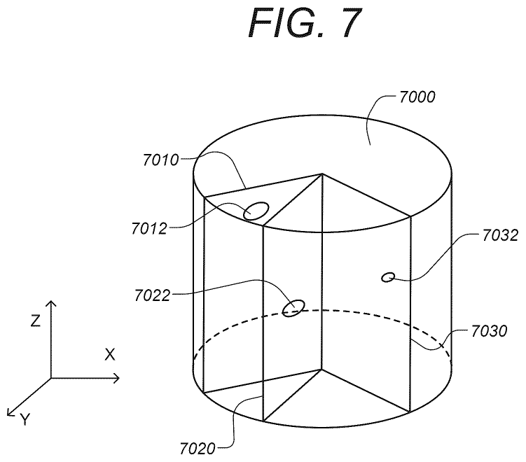

FIG. 8 shows a screen shot during the scanning of a supine patient with a 15 cm diameter prototype model of a Rotary ABUS as described in this patent specification, operating at 8.9 MHz and 39 fps. The image shows good, over 7 cm ultrasound penetration, passing through the chest wall at 4.5 cm.

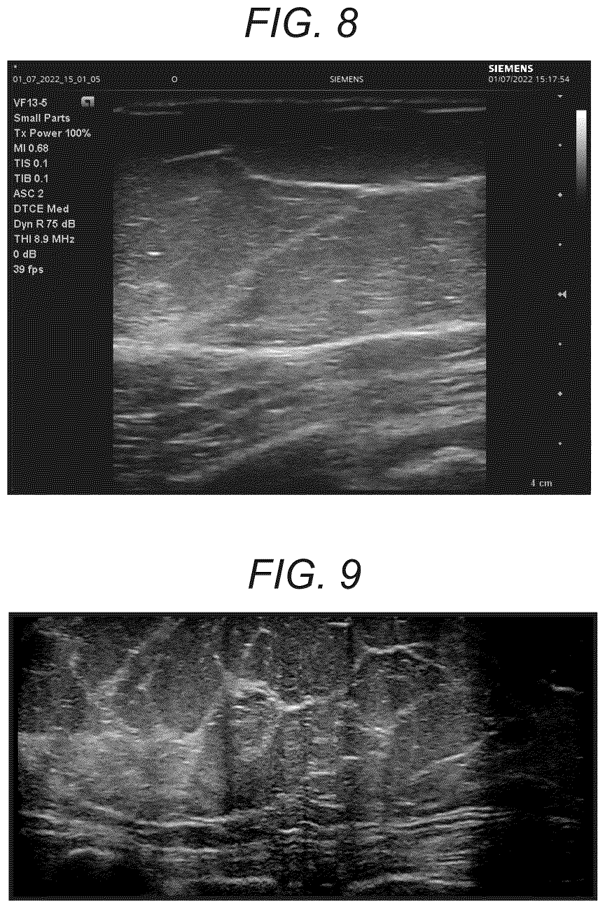

FIG. 9 shows a screen shot of two 2D images that were acquired 180° apart (approx. 30 seconds apart) such that ends of the images that are at the scanning center are next to each other. The center seam, where the 2D images meet, is barely visible, which shows that during the 30 second interval, the breast was held sufficiently firm with the scanner described in this patent specification.

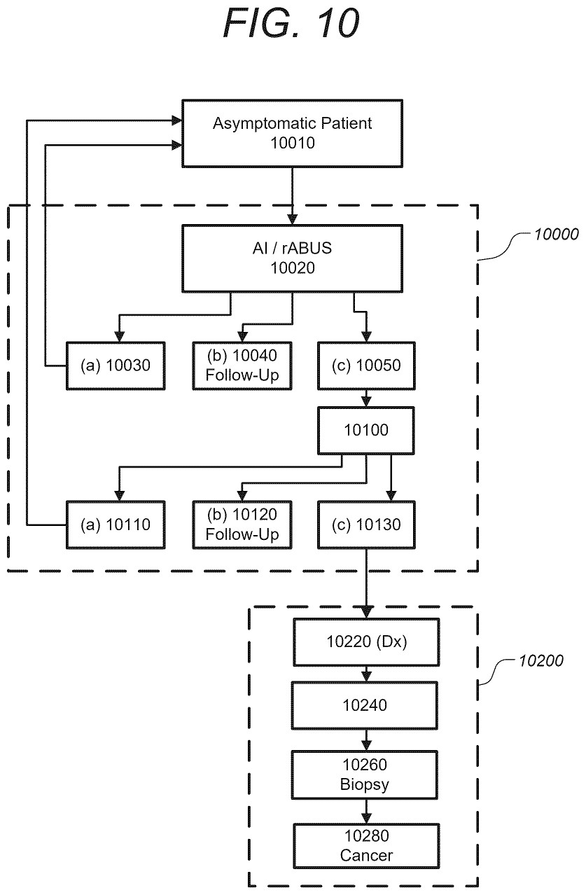

According to one embodiment, referring to FIG. 10, an asymptomatic patient 10010 schedules or walks into an AI-centric physician-assisted screening center 10000, and gets a screening of both breasts with an AI/rABUS (Rotary ABUS) system 10020. The rABUS scans each breast and obtains a total of approx. 1,000-2000 2D images of each breast. AI and preferably advanced AI (AII) is applied to the 2D images at step 10020.

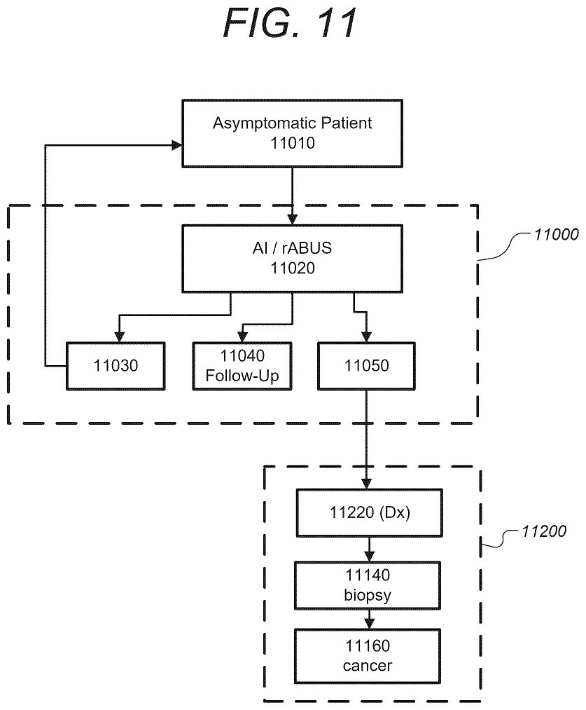

AI as used in this application means AI applied to processing ultrasound results for notional 2D slices produced from the rABUS system described in this patent specification, or 3D results produced from the 2D results, to find suspicious locations. The AI can be the as currently available from QView Medical, adapted to carry out the pertinent steps described below regarding FIGS. 10 and 11, and/or as proposed in Shen et al (8) but adapted to carry out the pertinent steps of FIGS. 10 and 11, and/or as described in U.S. Pat. Nos. 9,498,184 and 11,439,362 but adapted to carry out the pertinent steps of FIGS. 10 and 11, and/or as described in a paper Luo L et al (11), and reference 35 therein (I. Goodfellow, Y. Bengio and A. Courville, Deep learning. MIT press, 2016), all of which are incorporated by reference herein.

Advanced AI (AII) as used in this patent specification refers such AI as improved to find apparent abnormalities, to estimate the respective 3D locations and volumes of these abnormalities (volumes of interest of VOI), and to compare VOIs from one patient examination with VOIs from a prior examination and estimate changes in volume of respective VOIs as described below, including for directing further actions of or for patients. Per AI principles, AAI can be continuously improved by adjustments based on comparing AAI results with results from observation such as of biopsies, surgery, etc. as known in AI principles.

The rABUS, under development currently, scans each breast just once and obtains a total of approx. 1000-2000 2D images. rABUS is thus able to improve facility throughput by up 3-fold or more over the rectilinear ABUS (such as GEHC Invenia, which scans rectilinearly along 3 or more different paths per breast). Furthermore, during re-screen on returning patients, rABUS can better re-locate the prior suspicious lesions or area, faster and more accurately, than a rectilinear ABUS due to its scanning geometry. If any incoming patient has a prior scan, the advanced AI can compare the current scan with the prior scan to see if there are any volume changes rather than just area changes of Volumes of Interest (VOIs) representing suspicious portions of the breast, and can determine the degree of change, e.g., volume growth of VOIs. Initially, before rABUS reaches the commercial stage, this advanced AI with temporal volume comparison algorithm can be used with the currently available rectilinear ABUS, except at a higher overall cost and slower patient throughput.

As illustrated in FIG. 10, the decision by AAI preferably categorizes the findings of VOIs into three groups: (a) not suspicious 10030, (b) follow-up 10040, and (c) suspicious 10050. The not suspicious patient 10030 is requested to return for screening, depending on patient's age, preferably in 6 months for under age 50 or a year for older patients or other appropriate periods. The follow-up patient 10040 is asked to return for re-examination in less time, for example 3-6 months. The follow-up patient 10040 is selected for example when a very small suspicious lesion (below 5-7 mm) is detected, and the patient has no prior recent scans. The results for the suspicious patient 10050, are reviewed by a physician reader 10100, who would divide the suspicious patients 10050 into 3 groups: (a) not suspicious 10110, (b) follow-up 10120, and (c) very suspicious 10130. The not suspicious patients 10110 can be directed to return in for example 3 months, 6 months or a year depending on the age group. The follow-up patients 10120, who in the physician reader's opinion is not suspicious enough to be sent to diagnostic workup examinations, can be asked to return for re-examination in say 3 to 6 months. The very suspicious patients 10130, who would have larger lesions or smaller lesions with clear and likely significant volume growths, can be directed to a physician operated facility 10200 for further diagnostic (Dx) workup procedures 10220, which can then lead to further actions 10240 such as additional imaging, follow-ups and biopsy 10260, leading to detection of cancers 10280. The physician reader 10100 in this case can operate remotely from the screening system via cloud or internet. The reading can be done at the end of screening day in batch reading.

The screening process can include computer-generating a patient report based on the AI assessment in step 10020 and or the physician assessment in step 10100, which report (i) directs a patient with a VOI assessed as likely cancerous to work-up medical procedures to further characterize or dismiss the likely cancerous VOI, (ii) directs a patient with a VOI assessed as follow-up to an ultrasound screening after a time interval related to the patient's age and medical history, and (ii) directs a patient with a VOI assessed as benign to an ultrasound screening after a time interval that also is related to the patient's age and medical history but typically is longer.

The decision point for suspicious AI decision is based on the operating point on the AI's ROC (receiver operator curve) where the AI sensitivity is equivalent or better to that of an experienced physician. At the present stage of development, QView's autonomous reading AI is potentially able to remove 67% of the cases, i.e., not suspicious 10030 patients, without further reading by physicians. Adding temporal volume comparison algorithm initially is estimated to increase the specificity (or benign removal rate) to 80% to 90%. At this point, each physician reader can have a patient capacity of 8,000 to 20,000, a 4-fold to 5-fold increase. Collection of many more ultrasound images of suspicious lesions, locations of breast quadrant the lesions are found, and cancer cases for further deep-learning training can further improve the performance of the advanced AI.

This removal rate improvement of advanced AI is expected to eventually reach a removal rate of 95% or better, and at that point no physician readers may be needed in the screening process, and rABUS plus advance AI can be able to broadly screen the millions of young dense breast women around the world, saving hundreds of thousand lives every year.

Referring to FIG. 11, an asymptomatic patient 11010 schedules or walks into an AI-centric, no-physician screening center 11000 to get a screening of both breasts with an advanced AI/rABUS system 11020.

The decision by AI categorizes the findings into 3 groups: (a) not suspicious 11030, follow-up 11040, and (c) suspicious 11050. The not suspicious patients 11030 are asked to return to screening, depending on patient's age, in say 6 months for under age 50 or a year for older patients. The follow-up patients 11040 is selected when a very small suspicious lesion (below 5-7 mm) is detected, and the patient has no prior recent scans. The follow-up patient may be directed to a return scan after an interval depending on age, which may be shorter than for not suspicious patients. The suspicious patients 11050 are directed to go to a physician operated facility 11200 for further diagnostic (Dx) workup procedures 11220, which could include additional imaging, follow-ups and biopsy 11140, and finally to cancer 11260.

The screening process of FIG. 11 can also involve computer-generating a patient report but in this case a report can be generated in step 11020, based on the AI assessment and without requiring a physician review of the characterization of VOIs, or with only spot review for some patents by a physician.

The AI decision point for not suspicious patients 11030 is based on the specificity point on the AI ROC where the AI sensitivity is equivalent or better to that of the average of practicing physician readers. The follow-up patient 11040 is selected when a very small suspicious lesion (below 5-7 mm) is detected, and the patient has no prior recent scans. The follow-up interval depends on the size of the small lesion and patient's age. The very suspicious patients 11050 can have a sensitivity of better than 90% and a specificity equal to or higher than 95%, which is equal or better than the physician readers' operating point.

The advanced AI can also benefit the physician reader in the diagnostic workup examinations. Typically, for each cancer detected in the under 50 age group, the diagnostic workup examination starts with as many as 25 to 50 patients. After the examination, there are maybe 3 to 8 very suspicious patients left for biopsy. As the advanced AI improved further, the number of very suspicious patients 11050, which show clear volume growths of VOIs, can be reduced to 3 to 8, so that the patients may be able to avoid the expensive and disruptive diagnostic workup examinations and proceed directly to biopsy.

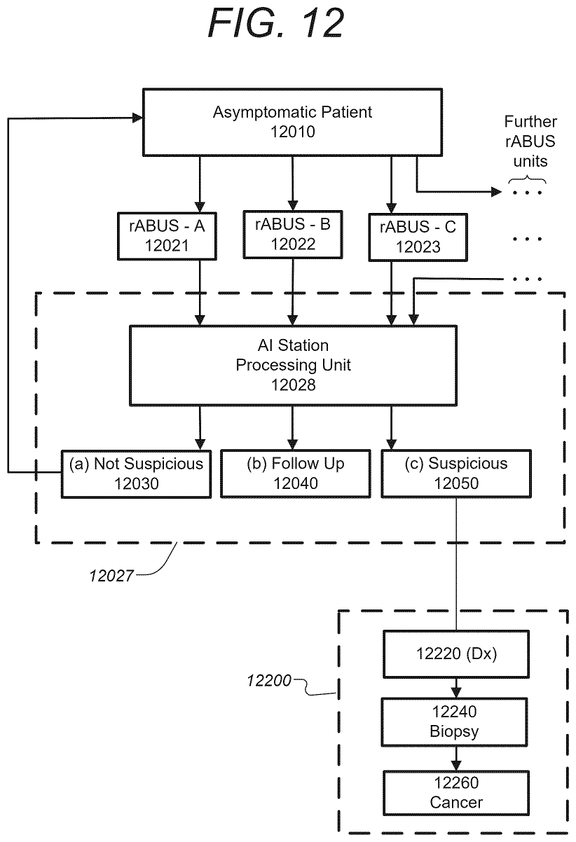

Referring to FIG. 12, a still lower cost approach, which shows three rABUS units 12021, 12022 and 12023, although many more units could be added, operating independently for the purpose of acquiring screening data from mainly asymptomatic patients (e.g. asymptomatic patient 12010).

The patient data are then analyzed instantaneously or by batches by a remote AI station 12027, consisting mainly of an AL processing unit 12028, connected to the rABUS units through wire or wireless means. The AI analysis results or findings are then sent back to the respective rABUS operators.

The decision by AI station 12027 categorizes the findings of each patient into three groups: (a) not suspicious 12030, (b) follow-up 12040, and (c) suspicious 12050. The not suspicious patients 12030 are asked to return to screening, depending on patient's age, in say 6 months for under age 50 or a year for older patients and the follow-up patients may be asked to return to screening sooner.

The screening process of FIG. 12 can also involve computer-generating a patient report but in this case a report can be generated in step 12020, based on the AI assessment and without requiring a physician review of the characterization of VOIs, or with only spot review for some patients by a physician.

The AI decision point for not suspicious patients 12030 is based on the specificity point on the AI ROC where the AI sensitivity is equivalent or better to that of the average of practicing physician readers.

The follow-up patient 12040 is selected when a very small suspicious lesion (below 5-7 mm) is detected, and the patient has no prior recent scans. The follow-up patient is asked to return for another rABUS screening and the interval depends on the size of the small lesion and patient's age, which could be 3 to 9 months.

The suspicious patients 12050 can have a sensitivity of better than 90% and a specificity equal to or higher than 95%, which is equal or better than the physician readers' operating point.

The suspicious patients 12050 are directed to go to a physician operated facility 12200 for further diagnostic (Dx) workup procedures 12220, which could include additional imaging, follow-ups and biopsy 12240, and finally to cancer 12260. As mentioned above, the advanced AI can also benefit the physician reader in the diagnostic workup examinations.

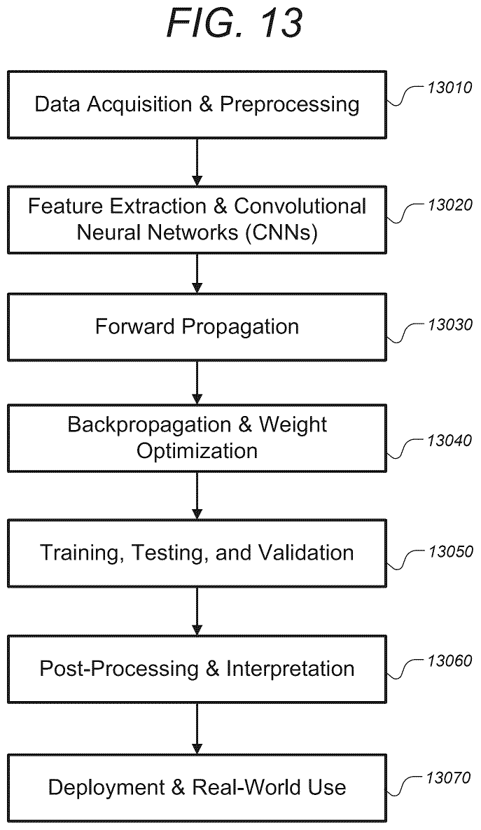

FIG. 13 is a block diagram illustrating how deep learning is used in medical imaging according to some embodiments. Deep learning is now widely used in medical image processing to improve diagnostics, automate image analysis, and enhance decision-making. FIG. 13 illustrates a typical step-by-step breakdown of how deep learning is applied in this field, incorporating concepts like backward projection, backpropagation, convolutional neural networks (CNNs), training, testing, and validation.

Data acquisition & preprocessing is shown in block 13010. Medical images are collected from various modalities like MRI, CT, X-rays, and ultrasound. These images undergo preprocessing, including noise reduction, normalization, and augmentation (rotation, scaling, etc.) to improve model performance.

Feature Extraction & Convolutional Neural Networks (CNN's) is shown in block 13020. CNNs are the commonly used deep learning models for image processing. They extract features such as edges, textures, and patterns using convolutional layers, which apply filters to detect relevant structures in the image. Pooling layers reduce dimensionality, making computations more efficient while retaining essential features.

Forward Propagation is shown in block 13030. The input image passes through multiple layers of the CNN, including convolutional layers, activation functions (like ReLU), pooling layers, and fully connected layers. The model makes an initial prediction (e.g., classifying an image as healthy or diseased).

Backpropagation & Weight Optimization is shown in block 13040. The error (loss) between the model's prediction and the actual label is calculated using a loss function (e.g., cross-entropy for classification, mean squared error for regression). Backpropagation is used to adjust the model's weights by computing the gradient of the loss function with respect to each weight using the chain rule. Gradient descent (or variants like Adam optimizer) updates the weights to minimize the loss.

Training, Testing, and Validation is shown in block 13050. Training: The model learns from a labeled dataset, adjusting weights iteratively using backpropagation. Validation: A separate validation set is used to tune hyperparameters and prevent overfitting. Testing: Once trained, the model is evaluated on an unseen test dataset to measure performance.

Post-Processing & Interpretation is shown in block 13060. Some models use backward projection techniques, often in medical imaging reconstruction (e.g., CT scan reconstruction from raw data). Explainability techniques like Grad-CAM (Gradient-weighted Class Activation Mapping) help visualize which regions of an image contributed to a model's decision. Deployment & Real-World Use is shown in block 13070.

Once validated, the model is deployed in clinical workflows, assisting radiologists by highlighting potential abnormalities, segmenting tumors, or enhancing images for better interpretation.

Deep learning has revolutionized medical imaging, significantly improving accuracy, efficiency, and early disease detection while reducing the workload of medical professionals.

According to some embodiments, systems such as Google's Medical Imaging Suite through Google Cloud can be used. The suite is designed to assist organizations in transforming imaging diagnostics by making imaging data accessible, interoperable, and useful. This suite provides services for imaging storage with the Cloud Healthcare API, allowing easy and secure data exchange using DICOM web. It also includes Imaging Lab, which helps automate the task of labeling medical images with AI-assisted annotation tools from MONAI.

Additionally, Google has developed Med-PaLM 2, a medical large language model that harnesses the power of Google's LLMs, aligned to the medical domain to more accurately and safely answer medical questions.

These offerings aim to support healthcare organizations in developing AI for imaging, facilitating faster and more accurate diagnosis of images, increasing productivity for healthcare workers, and improving access to better care and outcomes for patients.

Furthermore, NVIDIA offers hardware development kits that integrate their GPUs for medical image processing. One notable example is the NVIDIA Clara AGX Development Kit, designed to accelerate the development of AI-powered medical devices. This kit includes a Jetson AGX Xavier module, an RTX 6000 GPU, and other components to facilitate high-performance computing in medical applications. The Clara AGX SDK provides the necessary operating system, drivers, libraries, and example AI skills to support development.

Additionally, NVIDIA's Clara platform offers a range of tools and resources for medical imaging and device development, including software frameworks and hardware solutions tailored for healthcare applications.

While Google collaborates with NVIDIA to integrate AI-assisted annotation tools into its Medical Imaging Suite, the hardware development kits for medical image processing are primarily offered by NVIDIA.

A well-known 770+ page textbook on Deep Learning by Goodfellow, Bengio and Courville published by MIT Press (2016) is currently available free of charge online: www.deeplearningbook.org, and is hereby incorporated by reference. More specifically, there are several very good deep learning advanced AI reports on the rectilinear type ABUSs (automated breast ultrasound scanners). One example is that by Song et al (12), which is hereby incorporated by reference herein. Another is Indrakumari, Kumar, Murugan and Sherimon, Deep Learning in Medical Image Analysis, CRC Press 2025.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered as illustrative and not restrictive, and the body of work described herein is not to be limited to the details given herein, which may be modified within the scope and equivalents of the appended claims.

### LIST OF REFERENCES

1. Sung H, et al, Global Cancer Statistics 2020: GLOBOCAN Estimates of Incidence and Mortality Worldwide for 36 Cancers in 185Countries, CA CANCER J CLIN 2021; 71:209-249, doi: 10.3322/caac.21660. Available online at cacancerjournal.com
2. Webb M L, et al, A Failure Analysis of Invasive Breast Cancer, Cancer Month 00, 2013; DOI: 10.1002/cncr.28199
3. Monticciolo D L, Outcomes of Breast Cancer Screening Strategies Based on Cancer Intervention and Surveillance Modeling Network Estimates, Radiology 2024; 310 (2); e232658; https://doi.org/10.1148/radiol.232658.
4. Yala A, et al, A Deep Learning Model to Triage Screening Mammograms: A Simulation Study, Radiology 2019; 293:38-46;

https://doi.org/10.1148/radiol.2019182908
5. Peer P G M, Age-Dependent Growth Rate of Primary Breast Cancer, CANCER Jun. 1, 1993, Volume 71, No. 11.
6. Feig S A, et al, Risk, Benefit, and Controversies in Mammographic Screening, RSNA Categorical Course in Physics 1994; 121-137.
7. Sarkar S, et al, Evaluation of an Automatic Registration-Based Algorithm for Direct Measurement of Volume Change in Tumors, Int J Radiation Oncol Bio Phys, Vol 83, No 3, pp 1038-1046, 2012.
8. Shen Y, et al, Artificial Intelligence system reduces false-positive findings in the interpretation of breast ultrasound exams, Nature Communications 12, Article number 5645 (2021); NATURE COMMUNICATIONS|(2021) 12:5645 https://doi.org/10.1038/s41467-021-26023-2|www.nature.com/naturecommunications.
9. I. Goodfellow, Y. Bengio and A. Courville, Deep learning. MIT press, 2016; www.deeplearningbook.org.
10. Maglioretti et al, When Radiologists Perform Best: The Learning Curve in Screening Mammography Interpretation, Radiology Vol. 253, No. 3 (2009); http://pubs.rsna.org/doi/abs/10/radiol.2533090070.
11. Luo L et al, Deep Learning in Breast Cancer Imaging: A Decade of Progress and Future Directions, 2024, available at https://arxiv.org/html/2304.06662v4.
12. Song C, Chen X, Tang C, Xue P, Qiao Y, Artificial intelligence status prediction based on disease-specific images in the head and neck cancer: A systematic review and meta-analysis, Sep. 10, 2023, Journal of Medical Virology, https://doi.org/10.1002/jmv.29080.
13. Nature Communications (13) 12 article 5645 (2001); http://www.nature.com/articles/s4 1467-021-260023-2.
14. Pedemonte et al (14), in Radiology (2024).
15. Indrakumari, Kumar, Murugan and Sherimon, Deep Learning in Medical Image Analysis, CRC Press 2025.

1. An ultrasound system for breast cancer screening, comprising:
a rotary scanner comprising a rotary template, a radially oriented ultrasound transducer, a motor, and an acoustically transmissive membrane, wherein;
the rotary scanner is configured to scan a patient's breast by contacting a scan area of the breast with the template and transducer through the membrane and rotating the template and transducer with the motor relative to the membrane and the breast;
the rotary scanner has one or more breast-facing ribs and recesses between ribs and is configured to carry out the scan while exerting pressure on the breast through the membrane that is plural times lower per unit area at portions of the scan area remote from the transducer than under the transducer and further has an area that is relatively smooth and is configured to overlie the breast's nipple during the scan; and
the scan produces ultrasound results for a multiplicity of radially oriented two-dimensional (2D) planes in the breast;
a computer-implemented processor configured to process the ultrasound results by applying thereto algorithms configured to find one or more abnormalities and define them as three-dimensional (3D) volumes of interest (VOI) and to further divide the identified VOIs into (i) cancer-suspicious VOIs and (ii) at least one category of VOIs that are not cancer-suspicious, wherein the algorithms include comparing volumes of interest from a current scan with volumes of interest from one or more previous scans of the breast; and
a processing facility configured to receive at least ultrasound results for cancer-suspicious VOIs and to subject the ultrasound results for the cancer-suspicious VOIs to further computer processing and examination to identify cancerous VOIs.

2. The ultrasound system of claim 1, in which the one or more breast-facing ribs and recesses are circumferentially spaced from the transducer and each other, whereby breast portions facing the one or more recesses experience the lower pressure per unit area.

3. The ultrasound system of claim 2, in which the one or more recesses comprise plural recesses that are circumferentially spaced from the transducer and from each other by breast-facing ribs with rounded surfaces.

4. The ultrasound system of claim 2, in which the area of the template that is relatively smooth is free of the one or more ribs and recesses.

5. The ultrasound system of claim 2, in which the template is configured to rotate about a center of rotation spaced toward the patient's head from the breast's nipple.

6. The ultrasound system of claim 5, in which the center of rotation is at the upper outer quadrant of the breast being scanned.

7. The ultrasound system of claim 2, in which the breast-facing surface of the template converges to the center of rotation of the template at an angle of no more than 15 degrees.

8. The ultrasound system of claim 1, in which the membrane and template are uninterrupted by a nipple hole.

9. The ultrasound system of claim 1, in which the rotary scanner is configured to scan an upwardly facing breast.

10. The ultrasound system of claim 1, in which the transducer protrudes toward the breast from a majority of the breast-facing surface of the template, whereby breast portions that are in the scan area but are remote from the transducer experience said lower pressure per unit area.

11. The ultrasound system of claim 1, in which the computer-implemented processor is further configured to divide the VOIs that are not cancer-suspicious into (a) likely benign VOIs, (b) VOIs for follow-up.

12. The ultrasound system of claim 1, in which the computer-implemented processor is configured to carry out the dividing through an artificial intelligence process.

13. The ultrasound system of claim 1, in which the comparing of volumes include comparing the volumes of respective pairs of VOIs that match in 3D location in the breast.

14. The ultrasound system of claim 1, in which the rotary template is essentially flat at a surface therein facing the breast except for one or more shallow recesses that are spaced from the ultrasound transducer.

15. The ultrasound scanner of claim 1, including a ring-shaped support for the mesh and ring-shaped, foam-like material over a breast-facing surface of the ring-shaped support.

16. The ultrasound system of claim 1, in which the rotary scanner includes a window and both the window and the rotary template are sufficiently transparent to enable visualization of the breast from outside the rotary scanner.

17. The ultrasound system of claim 1, in which the rotary template is transparent and the rotary scanner includes an imaging module configured to view the breast through the rotary template and the system further includes a display configured to display images of the template and breast taken with the imaging module.

18. The ultrasound system of claim 1, in which the rotary template is shaped as a shallow dish converging to the center of rotation at an angle less than 15 degrees.

19. An ultrasound system for breast cancer screening, comprising:
a rotary scanner comprising a rotary template, one or more radially oriented ultrasound transducers, a motor, and an acoustically transmissive membrane, wherein;
the rotary scanner is configured to scan a patient's breast by contacting the breast with the template and the one or more transducers through the membrane and rotating the template and the one or more transducers with the motor relative to the membrane and the breast such that the one or more transducers maintain acoustic contact with the breast over a dish-shaped path;
the template has a breast-facing surface with one or more recesses from the dish-shaped path and an area that is free of the recesses and is configured to overly the breast's nipple while the template rotates over the breast; and
the scan produces ultrasound results for a multiplicity of two-dimensional (2D) planes in the breast;
a computer-implemented processor configured to process the ultrasound results by applying thereto algorithms configured to identify likely abnormalities as three-dimensional (3D) volumes of interest (VOI) and to divide the identified VOIs into (i) cancer-suspicious VOIs and (ii) at least one category of VOIs that are not cancer-suspicious; and
a processing facility configured to receive ultrasound results for cancer-suspicious VOIs and to subject the ultrasound results for the cancer-suspicious VOIs to further computer processing and observation to identify cancerous VOIs;
wherein at least one other computer-implemented processor and the processing facility compares volume of a VOI from a current scan with that of a similarly located VOI from a previous scan of the breast.

20. The ultrasound system of claim 19, in which the rotary template has one or more breast-facing rounded ribs that are circumferentially spaced from the transducer and from each other and define said one or more recesses, whereby breast portions facing the one or more recesses experience lower pressure per unit area than breast portions that are under the one or more transducers and ribs.

21. The ultrasound system of claim 19, in which a breast-facing area of the template that is free of the one or more recesses also is free of a nipple hole.

22. The ultrasound system of claim 19, in which the center of rotation of the template is at the upper outer quadrant of the breast being scanned.

23. The ultrasound system of claim 19, in which the breast-facing surface of the template converges to the center of rotation of the template at an angle of no more than 15 degrees.

24. The ultrasound system of claim 19, in which the algorithms that the computer-implemented processor is configured to apply are artificial intelligence algorithms.

25. The ultrasound scanner of claim 19, including a ring-shaped support for the mesh and ring-shaped, foam-like material over a breast-facing surface of the ring-shaped support.

26. The ultrasound system of claim 19, in which the patient's breast faces up during the scanning.

27. A method of ultrasound screening for breast cancer comprising:
scanning a patient's breast with a rotary template and one of more ultrasound transducers that together rotate over the breast while the one or more transducers maintain acoustic coupling with the breast through a membrane to produce two-dimensional (2D) ultrasound images for respective notional planes through the breast;
subjecting the 2D ultrasound images to an artificial intelligence (AI) process configured to identify likely abnormalities in the breast as volumes on interest (VOIs) and to assess the VOAs to produce AI results that characterize each as (i) benign VOI, (ii) VOI for follow-up, or (iii) likely cancerous VOI, wherein the AI process includes comparing a volume of one or more of the identified VOIs with the volume of a corresponding one or more VOI from one or more previous scan of the breast if available;
computer-generating the AI results to automatically produce a patient report that (i) directs a patient with a VOI assessed as likely cancerous to work-up medical procedures to further characterize the likely cancerous VOI, (ii) directs a patient with a VOI assessed as follow-up to an ultrasound screening after a time interval related to the patient's age and medical history and different for different age groups, and (iii) directs a patient with a VOI assessed as benign to an ultrasound screening after a time interval that also is related to the patient's age and medical history but is longer than for a patient with a benign VOI.

28. The method of claim 27, further including a physician review of ultrasound results relating to a VOI that the AI process has characterized as likely cancerous to confirm the VOI as likely cancerous or change its characterization to benign or follow-up, and wherein the step of computer-generating a patient report directs the patient based on the results produced in the physician review.

29. The method of claim 27, in which the computer-generating of a patient report is configured to direct patients to follow-up ultrasound screening based on a table relating patient age to statistical rate of doubling of VOI volumes.

30. The method of claim 27, in which the computer-generating of a patient report is configured to direct patients with a VOI for follow-up and are under 50 years of age to a follow-up ultrasound screening in less than 6 months.

31. A rotary scanner comprising a rotary template, a radially oriented ultrasound transducer, a motor, and an acoustically transmissive membrane, wherein;
the rotary scanner is configured to scan a patient's breast by contacting a scan area of the breast with the template and transducer through the membrane and rotating the template and transducer with the motor relative to the membrane and the breast;
the rotary scanner template has one or more breast-facing recesses and is configured to carry out the scan while exerting pressure on the breast through the membrane that is plural times lower per unit area at portions of the scan area remote from the transducer than under the transducer and further has a central area for nipple placement that is smoother than the area portion of the template with the recesses.

32. The rotary scanner of claim 31, in which the template is spherically curved, with a radius of curvature equal to 1-3 times the diameter of the rotary template.

33. The rotary scanner of claim 31, in which the rotary template is conically curved, with a less than 15° angle.

34. The rotary scanner of claim 31, in which the template surface facing the breast is concavely curved.
